(19) United States
(12) Patent Application Publication
(10) Pub. No.: US 2011/0269130 A1
(43) Pub. Date: Nov. 3, 2011

(54) Antibiotice Susceptibility Profiling Methods

(76) Inventors: Song Shi, Reistertown, MD (US); Ben Turng, Ellicott City, MD (US); Liping Feng, Cockeysville, MD (US)

(21) Appl. No.: 13/124,861

(22) PCT Filed: Oct. 23, 2009
(86) PCT No.: PCT/US09/61851

§ 371 Date: Jul. 22, 2011

Related U.S. Application Data
(60) Provisional application No. 61/108,427, filed on Oct. 24, 2008.

(51) Int. Cl. C12Q 1/68; C12Q 1/04

(52) U.S. Cl. 435/6.11; 435/34

(57) ABSTRACT

The invention provides methods for the rapid determination of the antibiotic susceptibility of a microorganism, such as, an infectious microorganism in a biological sample, using fluorescence in situ hybridization (“FISH”). Methods of the invention may be applied to the rapid identification, typing, antibiotic susceptibility determination, and/or antibiotic minimum inhibitory concentration (MIC) determination for any infectious microorganism, such as a Gram positive bacteria, a Gram negative bacteria, or a yeast.

### DESCRIPTION OF THE INVENTION

This application claims priority to U.S. Provisional Patent Application No. 61/108,427, filed on Oct. 24, 2008, the contents of which are incorporated herein by reference.

### FIELD OF THE INVENTION

The invention relates to a novel method for microorganism identification and antibiotic susceptibility testing. More specifically, this invention relates to the use of fluorescence in situ hybridization (“FISH”) for the rapid quantitative identification and drug susceptibility screening of infectious microorganisms present in clinical specimens.

### BACKGROUND OF THE INVENTION

Microorganism infections, such as bacteremia, sepsis, and pneumonia, are frequently associated with multi-drug-resistant organisms (MDRO). According to the Centers for Disease Control and Prevention, MDROs are defined as microorganisms that are resistant to three or more classes of antimicrobial agents. Rapid and accurate methods of microorganism identification and drug susceptibility testing are essential for disease diagnosis, treatment of infection, and to trace disease outbreaks associated with microbial infections.

Traditional methods of microorganism identification involve conventional microbiological procedures (i.e., isolating a pure colony of the microorganism in question and then culturing that isolate on solid medium or in liquid phase) followed by analysis of the biochemical and/or phenotypic characteristics of the organism (i.e., gram staining and/or DNA analysis). Traditional methods of drug susceptibility testing typically require the isolation of a pure colony of the microorganism in question and then analysis of the growth of that isolate using a broth dilution or agar diffusion assay.

The broth dilution method involves inoculating a pure isolate of the microorganism in question into a growth medium (typically, Mueller Hinton broth) containing a series of predetermined concentrations of the particular antibiotic for which a minimum inhibitory concentration (MIC), or an MIC-like measurement, is to be determined. The medium may also contain a chromogenic or fluorogenic enzyme substrate which, when metabolized by the microorganism, releases a moiety that imparts a color or other detectable change to the medium. See, e.g., U.S. Pat. Nos. 4,925,789; 5,620,865; 6,387,650; 6,472,167; and 4,591,554, the entire contents of which are incorporated by reference. The inoculated medium is incubated for 18-24 hours and observed for visible growth, as measured by turbidity, pellet size, and/or release of the chromogenic or fluorogenic moiety. The lowest antibiotic concentration that completely inhibits visible growth of the isolated organism is recorded as the MIC.

The agar diffusion assay involves the placement of an antibiotic containing disc or an antibiotic gradient strip on the surface of an agar medium (typically, a Mueller Hinton agar plate) that has been inoculated with a pure isolate of the microorganism in question. The medium may also contain a chromogenic or fluorogenic enzyme substrate which, when metabolized by the microorganism, releases a moiety that imparts a color or other detectable change to the medium. See, e.g., U.S. Pat. Nos. 4,925,789; 5,620,865; 6,387,650; 6,472,167; and 4,591,554, the entire contents of which are incorporated by reference. The plates are incubated for 18-24 hours, during which time the antibiotic substance diffuses away from the disc or strip, such that the effective concentration of antibiotic varies as a function of the radius from the disc or strip. The diameter of the resulting area of no growth and/or no color (i.e., the zone of inhibition) around the disc or strip, if any, is directly proportional to the MIC.

Current FDA-approved methods for antibiotic susceptibility testing require inoculation of around 10⁵ CFU/mL microorganisms. Because clinical samples generally contain substantially less than 10⁵ CFU/mL, it is difficult to apply FDA-approved tests directly to clinical specimens. Typically, clinical samples are inoculated into culture medium and grown until the number of microorganisms reach about 10⁸ CFU/mL. Usually, the processes of microorganism identification and antibiotic susceptibility testing require 48 to 72 hours to be completed, during which time the microorganism continues to spread in the patient and in the environment. Shortening the time necessary to identify the infectious microorganism and select an effective antibiotic regimen could significantly decrease morbidity and mortality rates, prevent epidemic outbreaks, and reduce the cost of treating patients with aggressive microorganism infections.

Accordingly, a primary object of the invention is to provide a method for rapid microorganism detection and drug susceptibility screening. This object is achieved by using FISH to identify microorganisms and screen for their drug susceptibility directly from clinical specimens. This object is alternatively achieved by using FISH to identify microorganisms and screen for their drug susceptibility directly from culture specimens, where the microorganism in a clinical specimen has been enriched to a detectable scale by growing in a growth medium.

### SUMMARY OF THE INVENTION

In one aspect, the invention provides methods of detecting the antibiotic susceptibility of a microorganism in a sample by dividing the sample into a plurality of subsamples, and then contacting each subsample with growth media having a different antibiotic compound and/or a different antibiotic concentration so that the susceptibility of the microorganism to different antibiotic compounds, and/or different concentrations of a given antibiotic compound, can be determined. The method further includes the steps of growing the antibiotic resistant microorganism contained in each antibiotic-containing subsample, and detecting the presence of a grown antibiotic resistant microorganism in each antibiotic-containing subsample by contacting the subsample with at least one fluorescent in situ hybridization (FISH) probe that hybridizes to an antibiotic resistant microorganism. By this method of the invention, the presence of a grown antibiotic resistant microorganism in the subsample indicates that the microorganism is not susceptible to the antibiotic compound or antibiotic concentration present in the subsample, while the absence of a grown antibiotic resistant microorganism in the subsample indicates that the microorganism is susceptible to the antibiotic compound or antibiotic concentration present in the subsample. Accordingly, the method provides for the detection of an antibiotic susceptibility of a microorganism in a test sample.

The method of the invention optionally includes the use of a positive control subsample containing the (one or more) microorganism in the sample and the growth medium, but omitting any antibiotic compound. The microorganism present in the positive control subsample will grow in the growth medium regardless of its antibiotic susceptibility, and thereby serves as a positive control for the identification of the microorganism and for the ability of the growth medium to support the growth of the microorganism(s) in the sample.

In certain embodiments, the sample is a biological fluid obtained from a subject (e.g., a patient). Exemplary samples for use in the invention include bronchioalveolar lavages, bronchial washes, pharyngeal exudates, tracheal aspirations, blood samples, serum samples, plasma samples, lymph samples, cerebrospinal fluids, pleural fluids, deep needle aspirations, sputum samples, urine samples, nasal secretions, tears, bile samples, ascites fluid samples, pus, synovial fluids, vitreous fluids, vaginal secretions, and urethral secretions. In further embodiments, the sample used may be a culture fluid or specimen in which a body fluid or tissue extract from the subject has been incubated with a growth medium.

In further embodiments, the microorganism in the test sample may be a bacterium, such as Staphylococcus, Enterococcus, Pseudomonas aeruginosa, Escherichia coli, Klebsiella pneumoniae, Acinetobacter baumannii, Streptococcus pneumoniae, Stenotrophomonas maltophilia, Burkholderia cepacia, or Ralstonia pickettii. In certain embodiments, the microorganism is a Methicillin Resistant Staphylococcus aureus (MRSA). In further embodiments, the microorganism may be a yeast, such as a Candida species like Candida albicans, C. glabrata, C. tropicalis, C. krusei, C. parapsilosis, C. bracarensis, C. guilliermondii, C. lusitaniae, or C. dubliniensis.

In further embodiments, the antibiotic compound applied to the microorganism in the test sample may be amikacin, amoxicillin, amoxicillin/clavulanate, ampicillin, ampicillin/sulbactam, arbekacin, azithromycin, aztreonam, cefaclor, cefazolin, cefdinir, cefditoren, cefetamet-pivoxil, cefixime, cefmetazole, cefoperazone, cefoperazone/sulbactam, cefotaxime, cefotetan, cefotiam, cefoxitin, cefpirome, cefpodoxime-proxetil, cefsulodin, ceftazidime, ceftibuten, ceftizoxime, ceftriaxone, cefuroxime sodium, cephalexin, cephalothin, cerepime, chloramphenicol, ciprofloxacin, clarithromycin, clindamycin, colistin, daptomycin, ertapenem, erythromycin, fosfomycin, fusidic acid, garenoxacin, gatifloxacin, gemifloxacin, gentamicin, mupirocin, isepamycin, kanamycin, levofloxacin, lincomycin, linezolid, imipenem, lomefloxacin, meropenem, minocycline, moxalactam, moxifloxacin, mupirocin, nalidixic acid, netilmicin, nitrofurantoin, norfloxacin, ofloxacin, oxacillin, pefloxacin, penicillin G, piperacillin, pristinamycin, quinupristin, dalfopristin, rifampin, streptomycin, teicoplanin, telithromycin, temocillin, tetracycline, ticarcillin, ticarcillin/clavulanate, tobromycin, trimethoprim, trimethoprim/sulfamethoxazole, trimethoprim/sulfamethoxazole, or vancomycin. In other embodiments, the antibiotic compound may be fluconazole, itraconazole, or flucytosine.

In still further embodiments, the FISH probe includes a peptide nucleic acid (PNA), a locked nucleic acid (LNA), a deoxyribonucleic acid, or a ribonucleic acid, e.g., one that is complementary to a microorganism target sequence. In preferred embodiments, the FISH probe includes a fluorophore to facilitate detection. In further preferred embodiments, the FISH probe hybridizes to a ribosomal RNA of the microorganism. In certain embodiments, the FISH probe hybridizes to a genus or species specific nucleic acid sequence of the microorganism and thereby identifies the genus or species of the antibiotic resistant microorganism.

In further preferred embodiments, the method of the invention incorporates a plurality of genus or species specific fluorescent in situ hybridization (FISH) probes having distinguishable labels. The plurality of FISH probes are contacted with the subsample and the fluorescence of the FISH probe that hybridizes to the antibiotic resistant microorganism identifies the genus or species of the antibiotic resistant microorganism present in the subsample. This preferred embodiment thereby allows simultaneous positive identification of one or more microorganisms in the sample with the detection of an antibiotic susceptibility of each of the microorganisms.

In another preferred embodiment of this aspect, the method of the invention further includes the steps of determining the general type of microorganism(s) present in the sample, and then, based upon the type of microorganism(s) determined to be present in the sample, selecting an appropriate panel of suitable antibiotic compounds and/or different antibiotic concentrations for subsequent antibiotic susceptibility testing. The step of determining the type of microorganism(s) present in the sample may be performed by any technique known to those skilled in the art. For example, in some embodiments, the identification step may be performed by Gram staining or other biochemical reaction methods, polymerase chain reaction, mass spectrometry, and/or hybridization to a family, genus, or species specific fluorescent in situ hybridization (FISH) probe. Suitable panels of antibiotics to be screened for different microorganisms, e.g., Gram negative, Gram positive, and Streptococcus bacteria, are known in the art.

In yet another preferred embodiment of this aspect, a series of different concentrations of an antibiotic is contacted with the microorganism(s) in the subsamples and the minimum inhibitory concentration (MIC) of the antibiotic is determined to be the lowest antibiotic concentration that inhibits the growth of the microorganism in the subsample. Accordingly, the MIC for one or more antibiotics can be determined for each of the one or more microorganisms present in the sample, thereby facilitating, e.g., the appropriate selection of an antibiotic therapy in the instance where the sample is from a patient in need of treatment for an infection.

In another aspect, the invention provides methods of both identifying the microorganism in a sample and detecting its antibiotic susceptibility. In this aspect, the method of the invention includes dividing the sample into a plurality of subsamples, and using one or more of the subsamples to detect the type of microorganism present in the sample. The type of microorganism present in the sample may, optionally, be used to inform the selection of one or more of the antibiotic compounds to be tested to determine whether the microorganism is susceptible to it (e.g., through the selection of an appropriate antibiotic panel based upon the Gram positive or Gram negative character of a bacterial microorganism present in the sample). One or more of the subsamples are contacted with growth media having a different antibiotic compound and/or a different antibiotic concentration, optionally adapted to the type of microorganism present, so that the susceptibility of the microorganism to different antibiotic compounds, and/or different concentrations of a given antibiotic compound, can be determined. The method further includes the steps of growing the antibiotic resistant microorganisms present in each antibiotic-containing subsample, and then detecting the presence of grown antibiotic resistant microorganism in each antibiotic-containing subsample by contacting the subsample with at least one fluorescent in situ hybridization (FISH) probe that hybridizes to an antibiotic resistant microorganism. Accordingly, by this method, the identification step is used to determine the microorganism present in the sample, and the presence of grown antibiotic resistant microorganisms in the subsample indicates that the microorganism is not susceptible to the antibiotic compound or concentration present in the subsample, while the absence of grown antibiotic resistant microorganism in the subsample indicates that the microorganism is susceptible to the antibiotic compound or concentration present in the subsample.

The step of determining the type of microorganism(s) present in the sample may be performed by any technique known to those skilled in the art. For example, in some embodiments, the identification step may be performed by Gram staining or other biochemical reaction methods, polymerase chain reaction, mass spectrometry, and/or hybridization to a family, genus, or species specific fluorescent in situ hybridization (FISH) probe.

The method of the invention optionally includes the use of a positive control subsample containing the (one or more) microorganism in the sample and the growth medium, but omitting any antibiotic compound. The microorganism present in the positive control subsample will grow in the growth medium regardless of its antibiotic susceptibility, and thereby serves as a positive control for the identification of the microorganism and for the ability of the growth medium to support the growth of the microorganism(s) in the sample.

In certain embodiments of this aspect of the invention, the sample is a biological fluid obtained from a subject (e.g., a patient). Exemplary samples for use in the invention include bronchioalveolar lavages, bronchial washes, pharyngeal exudates, tracheal aspirations, blood samples, serum samples, plasma samples, lymph samples, cerebrospinal fluids, pleural fluids, deep needle aspirations, sputum samples, urine samples, nasal secretions, tears, bile samples, ascites fluid samples, pus, synovial fluids, vitreous fluids, vaginal secretions, and urethral secretions. In further embodiments, the sample used may be a culture fluid or specimen in which a body fluid or tissue extract from the subject has been incubated with a growth medium.

In further embodiments of this aspect of the invention, the microorganism in the test sample may be a bacterium, such as Staphylococcus, Enterococcus, Pseudomonas aeruginosa, Escherichia coli, Klebsiella pneumoniae, Acinetobacter baumannii, Streptococcus pneumoniae, Stenotrophomonas maltophilia, Burkholderia cepacia, or Ralstonia pickettii. In certain embodiments, the microorganism is a Methicillin Resistant Staphylococcus aureus (MRSA). In further embodiments, the microorganism may be a yeast, such as a Candida species like Candida albicans, C. glabrata, C. tropicalis, C. krusei, C. parapsilosis, C. bracarensis, C. guilliermondii, C. lusitaniae, or C. dubliniensis.

In further embodiments of this aspect of the invention, the antibiotic compound applied to the microorganism in the test sample may be amikacin, amoxicillin, amoxicillin/clavulanate, ampicillin, ampicillin/sulbactam, arbekacin, azithromycin, aztreonam, cefaclor, cefazolin, cefdinir, cefditoren, cefetamet-pivoxil, cefixime, cefmetazole, cefoperazone, cefoperazone/sulbactam, cefotaxime, cefotetan, cefotiam, cefoxitin, cefpirome, cefpodoxime-proxetil, cefsulodin, ceftazidime, ceftibuten, ceftizoxime, ceftriaxone, cefuroxime sodium, cephalexin, cephalothin, cerepime, chloramphenicol, ciprofloxacin, clarithromycin, clindamycin, colistin, daptomycin, ertapenem, erythromycin, fosfomycin, fusidic acid, garenoxacin, gatifloxacin, gemifloxacin, gentamicin, mupirocin, isepamycin, kanamycin, levofloxacin, lincomycin, linezolid, imipenem, lomefloxacin, meropenem, minocycline, moxalactam, moxifloxacin, mupirocin, nalidixic acid, netilmicin, nitrofurantoin, norfloxacin, ofloxacin, oxacillin, pefloxacin, penicillin G, piperacillin, pristinamycin, quinupristin, dalfopristin, rifampin, streptomycin, teicoplanin, telithromycin, temocillin, tetracycline, ticarcillin, ticarcillin/clavulanate, tobromycin, trimethoprim, trimethoprim/sulfamethoxazole, trimethoprim/sulfamethoxazole, or vancomycin. In other embodiments, the antibiotic compound may be fluconazole, itraconazole, or flucytosine.

In still further embodiments, the FISH probe includes a peptide nucleic acid (PNA), a locked nucleic acid (LNA), a deoxyribonucleic acid, or a ribonucleic acid, e.g., one that is complementary to a microorganism target sequence. In preferred embodiments, the FISH probe includes a fluorophore to facilitate detection. In further preferred embodiments, the FISH probe hybridizes to a ribosomal RNA of the microorganism.

In further preferred embodiments, the method of the invention incorporates a plurality of genus or species specific fluorescent in situ hybridization (FISH) probes having distinguishable labels. The plurality of FISH probes are contacted with the subsample and the fluorescence of the FISH probe that hybridizes to the antibiotic resistant microorganism identifies the genus or species of the antibiotic resistant microorganism present in the subsample. This preferred embodiment thereby allows simultaneous positive identification of one or more microorganisms in the sample with the detection of an antibiotic susceptibility of each of the microorganisms.

In yet another preferred embodiment of this aspect, a series of different concentrations of an antibiotic is contacted with the microorganism(s) in the subsamples and the minimum inhibitory concentration (MIC) of the antibiotic is determined to be the lowest antibiotic concentration that inhibits the growth of the microorganism in the subsample. Accordingly, the MIC for one or more antibiotics can be determined for each of the one or more microorganisms present in the sample, thereby facilitating, e.g., the appropriate selection of an antibiotic therapy in the instance where the sample is from a patient in need of treatment for an infection.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate one (several) embodiment(s) of the invention and together with the description, serve to explain the principles of the invention.

### BRIEF DESCRIPTION OF THE DRAWINGS

FIG. 1 is a schematic representation of one embodiment of the invention. “S” indicates antibiotic susceptibility; “I” indicates intermediate susceptibility; and “R” indicates antibiotic resistance.

FIG. 2 shows the detection of S. aureus by using PNA-FISH on filter membranes. S. aureus ATCC 29213 was identified as bright-green fluorescent cocci on the filter membranes. Images were taken with a FITC filter.

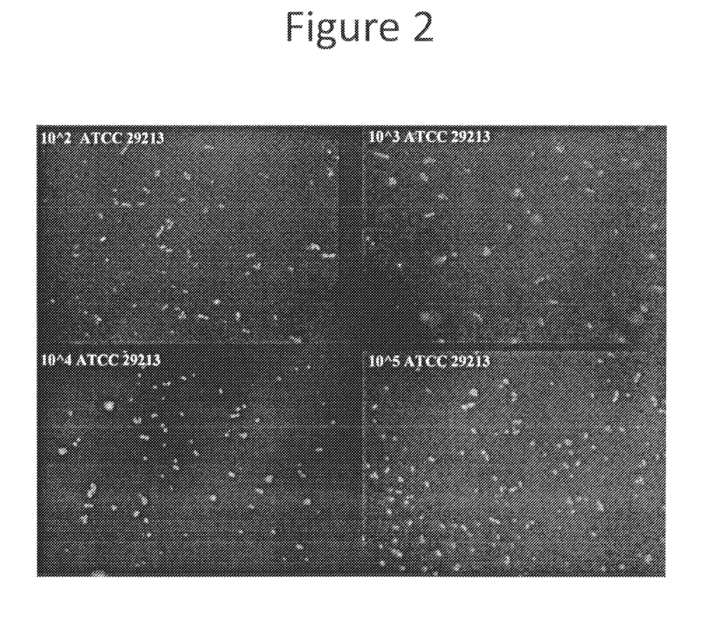

FIG. 3 shows the effect of oxacillin (OX) on the growth of wild type S. aureus ATCC 29213. The presence of OX killed the bacteria and the dead organisms were enlarged compared to their normal size.

FIG. 4 shows the effect of oxacillin (OX) on the growth of resistant S. aureus POS 3633. The presence of OX, even at high concentrations, had no effect on the growth of the resistant bacteria.

### DETAILED DESCRIPTION OF THE INVENTION

As used herein, the term “antibiotic susceptibility testing” refers to any test or assay for evaluating microorganisms for their susceptibility to antibiotics of interest. An antibiotic susceptibility test may be used to determine the clinical efficacy of an antibiotic for treating infection caused by a microorganism.

As used herein, the terms “susceptible” and “antibiotic susceptibility” indicate that the growth of a microorganism is inhibited by the usually achievable concentrations of an antimicrobial agent when the recommended dosage is used.

As used herein, the terms “intermediate” and “intermediate susceptibility” indicate that at the minimum inhibitory concentration (MIC) of an antimicrobial agent, which approaches usually attainable blood and tissue levels, growth of a microorganism is higher than for susceptible microorganisms. Intermediate susceptibility indicates clinical efficacy in body sites where the antimicrobial agents are physiologically concentrated or when a higher than normal dosage can be used.

As used herein, the terms “resistant” and “antibiotic resistance” indicate that microorganism growth is not inhibited by the usually achievable concentrations of the agent with normal dosage schedules and clinical efficacy of the agent against the microorganism has not been shown in treatment studies. These terms also indicate situations in which the microorganisms exhibit specific microbial resistance mechanisms.

As described above, this invention relates to the use of FISH for rapid and quantitative microorganism identification and drug susceptibility screening directly from clinical specimens. FISH allows the visualization of prokaryotic cells in their natural environment without cultivation. Thus, the method of the invention advantageously avoids the time-consuming steps associated with prior art methods in which clinical samples must be inoculated into culture medium and grown until FDA-approved levels of microorganisms are obtained, or in which individual clones of the microorganism in question must be isolated and those individually cultured to obtain sufficient numbers of the microorganism for further analysis.

Briefly, according to the method of the invention, a clinical specimen suspected of containing a microorganism is divided into subsamples. The subsamples are inoculated into a series of growth mediums having different antibiotic compounds and/or different antibiotic concentrations. The subsamples are incubated under conditions permitting growth of any antibiotic resistant microorganisms present in the subsample. The cells are then fixed, permeabilized, and hybridized with nucleic acid probes labeled directly or indirectly labeled with a detectable agent. The subsamples may be analyzed, for example, by microscopy, flow cytometry, or solid phase cytometry, and the presence of a detectable signal indicates antibiotic resistance in that subsample.

In some embodiments, the subsamples are grown on the media containing antibiotics for about 1 to 24 hours. In other embodiments, the subsamples are grown on the media containing antibiotics for about 2 to 8 hours. In other embodiments, the subsamples are grown on the media containing antibiotics for about 2 to 5 hours.

The clinical samples of the invention may comprise one type of microorganism or may comprise multiple microorganisms (i.e., polymicrobial infections). In one embodiment of the invention, the clinical sample is analyzed directly (i.e., the patient's blood is drawn into a blood culture bottle and the contents of the blood culture bottle is divided into subsamples for further analysis). In other embodiments of the invention, the clinical sample is cultured before dividing into subsamples in order to amplify the number of microorganisms present in the sample. In both embodiments, the method of the invention eliminates the sub-culture steps required in prior art methods, in which blood samples are first plated onto agar medium and then incubated for 18-24 hours to yield isolated colonies. Thus, an advantage of the method of the invention is the ability to more rapidly identify and test the antimicrobial susceptibility of microorganisms in clinical samples.

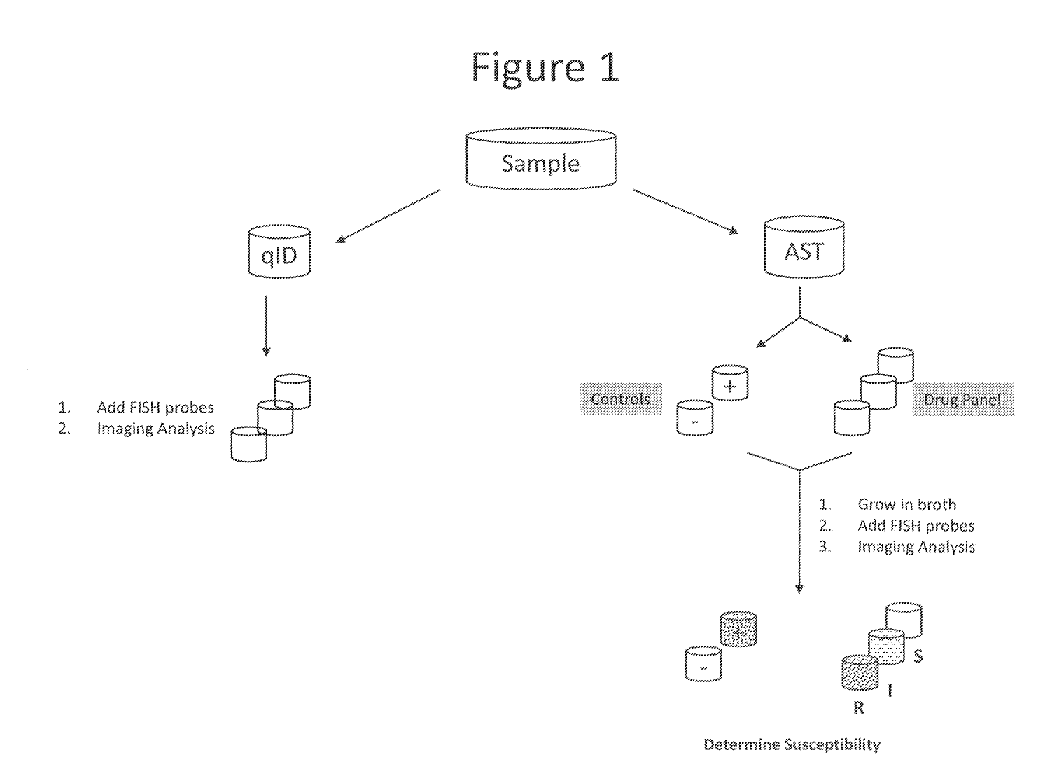

The method of the invention may optionally comprise a step of classifying or identifying the microorganism in the clinical specimen. FIG. 1 presents a schematic representation of this embodiment of the invention. Such an identification step may be performed by methods known to those skilled in the art, such as Gram staining or other biochemical reaction methods, polymerase chain reaction, mass spectrometry, and/or hybridization to a family, genus, or species specific fluorescent in situ hybridization (FISH) probe. If the microorganism present in the clinical specimen is identified before inoculating the subsamples into growth mediums having different antibiotic compounds and/or different antibiotic concentrations, a suitable panel of antibiotic compounds and/or concentrations can be selected for addition to the growth medium in the antibiotic resistance screen. For example, if the sample contains gram positive bacteria, a panel of antibiotic compounds and/or concentrations suitable for testing the antibiotic resistance of gram positive bacteria should be selected. Suitable panels and methods for antibiotic susceptibility testing (“AST”) of various types of microorganisms are known to those skilled in the art. For example, the Clinical and Laboratory Standards Institute (“CLSI”) publishes approved standards for various recommended AST methodologies for use with different types, genera, and species of microorganisms. See, for example, CLSI publications “Method for Dilution Antimicrobial Susceptibility Tests for Bacteria that Grow Aerobically; Approved Standard—Seventh Edition,” Vol. 26, No. 2 (January 2006), and “Performance Standards for Antimicrobial Disk Susceptibility Tests; Approved Standard—Ninth Edition,” Vol. 26, No. 1 (January 2006), which are incorporated by reference herein.

While determination of the composition of panels of antibiotic compounds and their concentrations for AST suitable for different classes of microorganisms is within the skill in the art, a non-inclusive list of antibiotics that may be used for this purpose, including representative concentration ranges presented as micrograms/milliliter (μg/ml) for Gram negative and positive bacteria, and Streptococcus species, is provided in the following table. In selecting the antibiotics forming a panel, typically one of skill in the art would select a number of antibiotics, for example, one to three, from each class of compounds.

| | | | Available Concentrations | | |
|---|---|---|---|---|---|
| Antimicrobic | | | Gram | Gram | |
| | | Drug | Negative | Positive | Strep |
| Class | Drug Name | Code | Range | Range | Range |
| 5-Fluoroquinolone | Ciprofloxacin | CIP | 0.12-4 | 0.12-4 | N/A |
| 5-Fluoroquinolone | Garenoxacin | GRN | 0.125-16 | 0.25-8 | 0.03-4 |
| 5-Fluoroquinolone | Gatifloxacin | GAT | 0.25-8 | 0.25-8 | 0.06-8 |
| 5-Fluoroquinolone | Gemifloxacin | GEM | 0.125-8 | 0.125-2 | 0.06-8 |
| 5-Fluoroquinolone | Levofloxacin | LVX | 0.25-8 | 0.25-8 | 0.25-16 |
| 5-Fluoroquinolone | Lomefloxacin | LOM | 0.25-8 | 0.25-8 | N/A |
| 5-Fluoroquinolone | Moxifloxacin | MXF | 0.12-8 | 0.12-8 | 0.06-8 |
| 5-Fluoroquinolone | Norfloxacin | NOR | 0.25-16 | 0.25-16 | N/A |
| 5-Fluoroquinolone | Ofloxacin | OFX | 0.25-8 | 0.25-8 | 0.5-16 |
| 5-Fluoroquinolone | Pefloxacin | PEF | 0.25-810 | 0.25-8 | N/A |
| Aminoglycoside | Amikacin | AN | 0.5-64 | 0.5-64 | N/A |
| Aminoglycoside | Arbekacin | ARB | 0.25-16 | 0.25-16 | N/A |
| Aminoglycoside | Gentamicin | GM | 0.5-16 | 0.5-16 | N/A |
| Aminoglycoside | Gentamicin- | GMS | N/A | 500 | 250-1000 |
| | Synergy | | | | |
| Aminoglycoside | Isepamycin | ISP | 0.5-32 | N/A | N/A |
| Aminoglycoside | Kanamycin | K | 0.5-64 | 0.5-64 | N/A |
| Aminoglycoside | Kanamycin- | KS | N/A | 250 | 250-1000 |
| | Synergy | | | | |
| Aminoglycoside | Netilmicin | NET | 0.5-32 | 0.5-32 | N/A |
| Aminoglycoside | Streptomycin- | STS | N/A | 1000 | 250-1000 |
| | Synergy | | | | |
| Aminoglycoside | Tobramycin | NN | 0.12-16 | 1-16 | N/A |
| Rifamycin | Rifampin | RA | N/A | 0.25-32 | N/A |
| B-Lac/B-Lac. Inh. | Amoxicillin/ | AMC | 0.5/0.25-32/16 | 0.25/0.12-32/16 | 0.125/0.06-32/16 |
| | Clavulanate | | | | |
| B-Lac/B-Lac. Inh. | Amoxicillin/ | AXC | 0.5/2-32/2 | 0.25/2-32/2 | N/A |
| | Clavulanate | | | | |
| B-Lac/B-Lac. Inh. | Ampicillin/ | SAM | 1/0.5-32/16 | 0.5/0.25-32/16 | N/A |
| | Sulbactam | | | | |
| B-Lac/B-Lac. Inh. | Ampicillin/ | SXA | 0.5/8-32/8 | 0.5/8-32/8 | N/A |
| | Sulbactam | | | | |
| B-Lac/B-Lac. Inh. | Cefoperazone/ | SCP | 0.5/8-64/8 | N/A | N/A |
| | Sulbactam | | | | |
| B-Lac/B-Lac. Inh. | Piperacillin/ | TZP | 0.5/4-128/4 | 1/4-128/4 | N/A |
| | Tazobactam | | | | |
| B-Lac/B-Lac. Inh. | Ticarcillin/ | TIM | N/A | 1/2-128/2 | N/A |
| | Clavulanate | | | | |
| B-Lactam Pen | Amoxicillin | AMX | 0.5-32 | 0.25-32 | 0.125-32 |
| B-Lactam Pen | Ampicillin | AM | 0.5-32 | 0.06-32 | 0.06-32 |
| B-Lactam Pen | Oxacillin | OX | N/A | 0.06-4 | N/A |
| B-Lactam Pen | Penicillin G | P | N/A | 0.06-32 | 0.03-8 |
| B-Lactam Pen | Piperacillin | PIP | 0.5-128 | 1-128 | N/A |
| B-Lactam Pen | Temocillin | TEM | 2-32 | N/A | N/A |
| B-Lactam Pen | Ticarcillin | TIC | 1-128 | 1-128 | N/A |
| Carbapenem | Ertapenem | ETP | 0.25-32 | 0.25-32 | 0.06-4 |
| Carbapenem | Imipenem | IPM | 0.25-16 | 0.5-16 | 0.015-4 |
| Carbapenem | Meropenem | MEM | 0.25-16 | 0.5-16 | 0.03-2 |
| Cephem | Cefacior | CEC | N/A | 0.5-32 | N/A |
| Cephem | Cefazolin | CZ | 0.5-32 | 0.5-32 | N/A |
| Cephem | Cefdinir | CDR | 0.12-4 | 0.12-4 | N/A |
| Cephem | Cefditoren | CDN | 0.125-8 | 0.125-8 | N/A |
| Cephem | Cefepime | FEP | 0.5-64 | 1-64 | 0.06-4 |
| Cephem | Cefetamet- | CAT | 0.25-16 | N/A | N/A |
| | pivoxil | | | | |
| Cephem | Cefixime | CFM | 0.125-8 | N/A | N/A |
| Cephem | Cefmetazole | CMZ | 2-64 | 1-64 | N/A |
| Cephem | Cefoperazone | CFP | 0.5-64 | 1-64 | N/A |
| Cephem | Cefotaxime | CTX | 0.5-64 | 1-64 | 0.06-4 |
| Cephem | Cefotetan | CTT | 2-64 | 1-64 | N/A |
| Cephem | Cefotiam | CFT | 0.5-64 | 0.5-64 | N/A |
| Cephem | Cefoxitin | FOX | 0.5-64 | 1-64 | N/A |
| Cephem | Cefpirome | CPO | 0.5-64 | 0.5-64 | N/A |
| Cephem | Cefpodoxime- | CPD | 0.12-8 | 0.5-8 | N/A |
| | proxetil | | | | |
| Cephem | Cefsulodin | CFS | 1-64 | N/A | N/A |
| Cephem | Ceftazidime | CAZ | 0.5-64 | 1-64 | N/A |
| Cephem | Ceftibuten | CTB | 0.5-32 | N/A | N/A |
| Cephem | Ceftizoxime | ZOX | 0.5-64 | 1-64 | N/A |
| Cephem | Ceftriaxone | CRO | 0.5-64 | 1-64 | 0.06-4 |
| Cephem | Cefuroxime | CXM | 1-64 | 1-64 | 0.12-4 |
| | sodium | | | | |
| Cephem | Cephalexin | CN | 1-64 | 0.5-64 | N/A |
| Cephem | Cephalothin | CF | 1-64 | 0.5-64 | N/A |
| Cephem | Moxalactam | MOX | 1-64 | 1-64 | N/A |
| Cyclic peptide | Colistin | CL | 0.5-4 | N/A | N/A |
| Folate Antagonist | Trimethoprim | TMP | 0.5-16 | 0.5-16 | N/A |
| Folate Antagonist | Trimethoprim/ | STG | 0.4/7.6-12.8/243.2 | 0.4/7.0-12.8/243.2 | N/A |
| | Sulfamethoxazole | | | | |
| Folate Antagonist | Trimethoprim/ | SXT | 0.5/9.5-16/304 | 0.5/9.5-16/304 | 0.06/1.19-16/304 |
| | Sulfamethoxazole | | | | |
| Glycopeptide | Teicoplanin | TEC | N/A | 0.5-32 | 1-32 |
| Glycopeptide | Vancomycin | VA | N/A | 0.5-32 | 0.06-32 |
| Ketolide | Telithromycin | TEL | N/A | 0.03125-8 | 0.06-4 |
| Lincosamide | Lincomycin | L | N/A | 0.5-16 | N/A |
| Macrolide | Azithromycin | AZM | N/A | 0.06-8 | N/A |
| Macrolide | Clarithromycin | CLR | N/A | 0.06-8 | N/A |
| Macrolide | Erythromycin | E | N/A | 0.125-8 | 0.015-4 |
| Monobactam | Aztreonam | ATM | 0.5-64 | N/A | N/A |
| Phenicol | Chloramphenicol | C | 1-32 | 1-32 | 1-32 |
| Lincosamide | Clindamycin | CC | N/A | 0.12-8 | 0.03-4 |
| Fusidane | Fusidic Acid | FA | N/A | 0.5-32 | N/A |
| Nitrofuran | Nitrofurantoin | FM | 8-512 | 16-512 | N/A |
| Oxazolidinone | Linezolid | LZD | N/A | 0.25-32 | 0.25-16 |
| Pseudomonic acid | Mupirocin | MUP | N/A | 0.0625-8 | N/A |
| Pseudomonic acid | High level | MUH | N/A | 256 | N/A |
| | Mupirocin | | | | |
| Quinolone | Nalidixic Acid | NA | 1-32 | N/A | N/A |
| Streptogramin | Pristinamycin | PR | N/A | 0.25-4 | 0.06-4 |
| Streptogramin | Quinupristin/ | SYN | N/A | 0.5-4 | 0.12-8 |
| | Dalfopristin | | | | |
| Tetracycline | Minocycline | MI | 0.5-16 | 0.5-32 | N/A |
| Tetracycline | Tetracycline | TE | 0.5-16 | 0.5-16 | 0.06-16 |
| Other | Beta-Lactamase | BL | N/A | Fixed | N/A |
| Other | ESBL | ESBL | Fixed | N/A | N/A |
| Other | Fosfomycin | FOS | 8-256 | 8-256 | N/A |
| Cyclic lipopeptide | Daptomycin | DAP | N/A | 0.125-32 | 0.03-16 |

BOLD = Different Range for Gram Negative and Gram Positive
Fixed = Fixed concentration defined by manufacturer

Suitable compounds for use in panels AST of yeast species, for example, Candida species, are known to those of skill in the art. Concentration ranges used in the panels typically will cover the susceptible, susceptible-dose dependent, intermediate, and resistant ranges. For example, three compounds that may be used in a panel for AST determination for yeasts are fluconazole (4-64 micrograms/ml), itraconazole (0.06-1 micrograms/ml), and flucytosine (2-32 micrograms/ml). Interpretative guidelines for in vitro AST of Candida species with these compounds appear in the following table.

| | | Susceptible- | | |
|---|---|---|---|---|
| | | Dose | | |
| Antifungal Agent | Susceptible | Dependent | Intermediate | Resistant |
| Fluconazole | ≦8 | 16-32 | — | ≧64 |
| Itraconazole | ≦0.125 | 0.25-0.5 | — | ≧1 |
| Flucytosine | ≦4 | — | 8-16 | ≧32 |

The clinical specimens that may be used in the method of the invention include, but are not limited to, tissue samples and biological fluids including bronchioalveolar lavage, bronchial washes, pharyngeal exudates, tracheal aspirations, blood, serum, plasma, lymph, cerebrospinal fluid, pleural fluid, deep needle aspirations, sputum, urine, nasal secretions, tears, bile, ascites fluid, pus, synovial fluid, semen, vitreous fluid, vaginal secretions, and urethral secretions from a human or veterinary patient. The clinical samples may be used directly in the method of the invention without additional culturing or subculturing steps. Alternatively, the clinical samples may be cultured before use in the method of the invention to increase the number of microorganisms in the sample.

The antibiotic compounds that may be used in the growth medium in the method of the invention include, but are not limited to, mikacin, amoxicillin, amoxicillin/clavulanate, ampicillin, ampicillin/sulbactam, arbekacin, azithromycin, aztreonam, cefaclor, cefazolin, cefdinir, cefditoren, cefetamet-pivoxil, cefixime, cefmetazole, cefoperazone, cefoperazone/sulbactam, cefotaxime, cefotetan, cefotiam, cefoxitin, cefpirome, cefpodoxime-proxetil, cefsulodin, ceftazidime, ceftibuten, ceftizoxime, ceftriaxone, cefuroxime sodium, cephalexin, cephalothin, cerepime, chloramphenicol, ciprofloxacin, clarithromycin, clindamycin, colistin, daptomycin, ertapenem, erythromycin, fosfomycin, fusidic acid, garenoxacin, gatifloxacin, gemifloxacin, gentamicin, mupirocin, isepamycin, kanamycin, levofloxacin, lincomycin, linezolid, imipenem, lomefloxacin, meropenem, minocycline, moxalactam, moxifloxacin, mupirocin, nalidixic acid, netilmicin, nitrofurantoin, norfloxacin, ofloxacin, oxacillin, pefloxacin, penicillin G, piperacillin, pristinamycin, quinupristin, dalfopristin, rifampin, streptomycin, teicoplanin, telithromycin, temocillin, tetracycline, ticarcillin, ticarcillin/clavulanate, tobromycin, trimethoprim, trimethoprim/sulfamethoxazole, trimethoprim/sulfamethoxazole, and vancomycin.

The available concentrations of antibiotics used in the method of the invention will vary based on the antibiotic compound chosen and the type of microorganism in the clinical sample. The concentrations used should cover the susceptible range, the susceptible-dose-dependent range, the intermediate range, and the resistant range. In some embodiments, the concentrations of antibiotics may range from about 0.1 (μg/ml) to about 300 (μg/ml). In other embodiments, the concentration of antibiotics may range from about 0.5 (μg/m) to about 100 (μg/ml). In further embodiments, the concentration of antibiotics may range from about 1 (μg/ml) to about 10 (μg/ml). Suitable concentrations of antibiotics are known in the art.

The probes used in the method of the invention may comprise deoxyribonucleic acids (DNA), ribonucleic acids (RNA), peptide nucleic acids (PNA), or locked nucleic acid (LNA). In certain preferred embodiments, the invention utilizes a FISH probe incorporating a PNA, which are DNA mimics with a pseudopeptide backbone. While DNA and RNA have a deoxyribose and ribose sugar backbone, respectively, the PNA backbone is composed of repeating N-(2-aminoethyl)-glycine units linked by peptide bonds. The various purine and pyrimidine bases are linked to the backbone by methylene carbonyl bonds. Since the backbone of PNA contains no charged phosphate groups, the binding between PNA/DNA strands is stronger than between DNA/DNA strands due to the lack of electrostatic repulsion. In other embodiments, the invention utilizes a FISH probe incorporating a LNA, which are a class of nucleic acid analogues in which the ribose ring is “locked” by a methylene bridge connecting the 2′-O atom with the 4′-C atom. LNAs contain the six common nucleobases (T, C, G, A, U and mC) that appear in DNA and RNA and thus are able to form base-pairs according to standard Watson-Crick base pairing rules. Oligonucleotides incorporating LNA have increased thermal stability and improved discriminative power with respect to their nucleic acid targets.

Typically, the probes will be from about 3 to about 300 nucleotides in length. More typically, the probes will be from about 10 to about 100 nucleotides in length. In particular embodiments, the probes are from about 1 to about 10 kb, or about 7 to about 15 kb, or about 10 to about 20 kb, or about 15 to about 30 kb, or about 20 to about 40 kb, or about 30 to about 50 kb, or about 40 to about 60 kb, or about 50 to about 70 kb, or about 60 to about 80 kb, or about 70 to about 90 kb, or about 80 to about 100 kb in length. In other embodiments, the probes are from about 5 to about 100 bp, or about 10 to about 50 bp, or about 7 to about 15 bp, or about 10 to about 25 bp, or about 15 to about 30 bp, or about 20 to about 40 bp, or about 30 to about 50 bp, or about 40 to about 60 bp, or about 50 to about 70 bp, or about 60 to about 80 bp, or about 70 to about 90 bp, or about 80 to about 100 bp in length. Methods for making suitable probes using, e.g., ribosomal nucleic acid subunit subsequence specific probes, are known to those skilled in the art. See, e.g., U.S. Pat. No. 5,612,183.

The probes used in the method of the invention may be specific for particular genetic loci and/or particular types of microorganisms. Such probes may be used singly or in combination with a set of probes specific for a different particular type of microorganism. For example, the probes may be specific for a particular antibiotic resistance gene and/or genetic loci. In other embodiments, the probes may be specific for gram positive or gram negative bacteria. In other embodiments, the probes are specific for a particular genus or species of microorganism. For example, the probes may be specific for bacteria including, but not limited to, Acinetobacter sp. (including Acinetobacter baumannii), Agrobacterium sp. (including Agrobacterium tumefaciens), Arthrobacter sp. (including Arthrobacter globiformis), Aquifex sp., Bacillus sp. (including Bacillus anthracis and Bacillus cereus), Brucella sp. (including Brucella melitensis, and Brucella suis), Burkholderia sp. (including Burkholderia cepacia, Burkholderia mallei and Burkholderia pseudomalle), Chlamydia sp., Chlorobium sp., Clostridium sp. (including Clostridium botulinum and Clostridium difficil), Desulfovibrio sp., Enterococcus sp., Escherichia sp. (including Escherichia coli), Flavobacterium sp., Flexibacter sp., Gloebacter sp., Helicobacterium sp. (including Helicobacter pylori), Klebsiella sp. (including Klebsiella pneumoniae), Lactococcus sp. (including Lactococcus lactis), Leptonema sp., Planctomyces sp., Pseudomonas sp. (including Pseudomonas aeruginosa and Pseudomonas putida), Ralstonia sp. (including Ralstonia pickettii), Rhizobium sp. (including Rhizobium loti and Rhizobium meliloti), Rhodocyclus sp., Staphylococcus sp., Stenotrophomonas sp. (including Stenotrophomonas maltophilia), Streptococcus sp. (including Streptococcus pneumoniae), Streptomyces sp. (including Streptomyces avermittilis and Streptomyces coelicolor), Synechococucus sp., Thermomicrobium sp., Thermus sp., Thermotoga sp., Xanthomonas sp. (including Xanthomonas axonopodis and Xanthomonas campestris), and Vibrio sp. (including Vibrio parahaemolyticus).

In a preferred embodiment, the probes are complementary to an rRNA, an mRNA, or a tRNA. In some embodiments, the probes are complementary to the 16S rRNA or 23S rRNA of the microorganism sought to be identified and/or tested for its antibiotic susceptibility. For example, an exemplary 16S rRNA sequence for detecting the bacteria Aeromonas, e.g., Aeromonas hydrophila, is the sequence GGAAGGTTGATGCC, or the sequence CGTATCAACTGTGACGT. Exemplary Acinetobacter FISH probe sequences are described in, for example, Carr, et al. (2003) Intl. J. Sys. Evolut. Microbiol. 53: 953-63; Wagner et al. (1994) Appl. Environ. Microbiol. 60: 792-800; and Snaidr, et al. (1997) Appl. Environ. Microbiol. 63: 2884-2896. Suitable family, genus, and/or species-specific FISH probes are known in the art or are readily designed based upon existing microorganism sequence information and hybridization design considerations. For example, In situ accessibility maps of 16S rRNA from E. coli, Pirellula sp. Strain 1 (Planctomycetes, Bacteria), Metallosphaera sedula (Crenarchaeota, Archaea), and Saccharomyces cerevisiae (Eucarya) for Cy3-labelled oligonucleotide probes have been determined (see Behrens et al. (2003) Appl. Environ. Microbiol. 69: 1748-58).

Exemplary sets of probes that are specific for particular types of bacteria include, but are not limited to, the probes provided in the S. aurens PNA FISH™ kit (AdvanDx, Woburn, Mass.), the E. coli/P. aeruginosa PNA FISH™ kit (AdvanDx, Woburn, Mass.), and the EK/P. aeruginosa PNA FISH™ kit (AdvanDx, Woburn, Mass.). Other exemplary sets of probes include those designed to hybridize to the 16S rDNA sequences of Acinetobacter strains B2, AB1110, 7N16, 4B02, 17A04, 9AoO1, and 4N13 (GenBank/EMBL/DDBJ accession numbers: AF509828, AF509823, AF509825, AF509827, AF509828, AF509829, and AF509830, respectively). Exemplary methods and design considerations for the use of fluorescent in situ hybridization for the identification and characterization of prokaryotes using FISH probes are well known in the art (see, e.g., Wagner et al. 2003) Curr. Opin. Microbiol. 6: 302-9).

In preferred embodiments, the invention incorporates a plurality of FISH probes (i.e., multiplex FISH) so that multiple types of infectious microorganisms can be identified by type (i.e., the general class of bacteria for the purpose of antibiotic panel selection) and/or for the purpose of identifying the microorganisms (e.g., by determining the family, genus, and/or species of the microorganism). The cocktail of FISH probes may comprise probes specific for different genetic loci and/or different microorganisms, or both. Such multiplex FISH analysis allows for identification of (1) the types of infectious microorganisms in a clinical sample (i.e., by identifying the general class of bacteria for the purpose of antibiotic panel selection); (2) the specific microorganisms in a clinical sample (e.g., by determining the family, genus, and/or species of the microorganism); and (3) the presence of specific genes in the microorganisms in the clinical sample. Methods for the simultaneous classification of individual bacterial cells within mixed populations have been developed using multispectral Bacterial Identification (mBID) technology, which utilizes a mixture of different fluorescent probes that are specific for 16S rRNA sequences of individual species of known bacteria (see, e.g., Tanner, et al. (2000) Biotechnology et alia 6: 1-9).

The principle of positional or space multiplexing may be applied to the method of the invention in order to expand the number of genera or species that are identified for any given sample by simply reusing each of the seven or more different fluorophores present in each FISH probe cocktail with distinct sets of genus or species-specific hybridization sequences and applying them to different subsamples. For example, a first FISH cocktail to simultaneously identify the presence of one or more of seven distinct Streptococcus species present in a subsample can be designed by linking each of, for example, the seven different fluorophores listed below, to seven different hybridization sequences, each sequence specific to a different species of Streptococcus. This cocktail is applied to a single subsample, and the identity of one or more of the FISH probes that hybridize to a sequence in the microorganism, identifies the Streptococcus species present in the sample, if any is present. The same set of seven different fluorophores may also then be linked to seven different hybridization sequences, each sequence specific to a different bacterial genus, such as Pseudomonas. This second FISH cocktail is applied to a second subsample, and the identity of one or more of the FISH probes that hybridize to a sequence in the microorganism identifies the Pseudomonas species present in the sample, if any. Therefore, the principle of positional or space multiplexing greatly expands the number of species or genera that can be identified in each sample, such that the number is limited only by the number of microorganism-containing subsamples that may be generated from a given sample. Notably, subculturing the original sample allows the number of microorganism subsamples to be expanded to virtually any number desired so that virtually any number of species or genera can be identified from each sample.

The probes used in the method of the invention may be labeled directly or indirectly with at least one detectable label. Appropriate detectable labels include, but are not limited to, enzymes, chromophores, fluorochromes (e.g., FITC or TRITC), and haptens (e.g., biotin or digoxigenin). When combinations of probes specific for different types of microorganisms are used in the method of the invention, the sets of probes are preferably labeled with different specific labels. Accordingly, exemplary fluorescent dye labels are those which emit at distinct wavelengths so that simultaneous detection of multiple microorganism types, families, genera, and/or species can be achieved. For example, multispectral identification of seven species can be achieved simultaneously utilizing the FISH probes linked to the following fluorescent dyes: Alexa 350 (emission filter 442 nm), Pacific Blue (emission filter 465 nm), Bodipy 493/503 (emission filter 520 nm), Bodipy R6G (emission filter 555 nm), Bodipy 564/570 (emission filter 585 nm), Bodipy 581/591 (emission filter 615 nm), Cy5 (emission filter 665 nm), and Cy5.5 (emission filter 700 nm) (see Tanner, et al. (2000) Biotechnology et alia 6: 1-9). Further suitable labels and methods for attaching such labels to probes are well known in the art.

The probe can be a molecular beacon type probe (see e.g., U.S. Pat. No. 7,422,852; and Xiaohang et al. (December 2000) Analyt. Chem., pgs. 747A-753A). In addition, the probe can be a florescence resonance energy transfer (FRET) type probe (see e.g., U.S. Pat. No. 7,282,331, the entire contents of which is incorporated by reference).

Various systems known in the art for enhancing or amplifying the signal may also be applied. Methods for increasing the sensitivity of FISH detection methods, including the use of multiple probes, helper oligonucleotides, PNA probes, treatment with chloramphenicol to increase rRNA content, in situ polymerase chain reaction amplification, bacterial chromosomal painting (BCP), enzymatic signal amplification (TSA-FISH), and polynucleotide probes and RING-FISH, are known in the art (see Zwirglmaier (2005) FEMS Microbiol. Lett. 246: 151-8).

The probes may be hybridized to the fixed and permeabilized samples using suitable conditions known to those skilled in the art. See, e.g., J. Sambrook, E. F. Fritsch, and T. Maniatis, Molecular Cloning: A Laboratory Manual, 2nd Ed., Cold Spring Harbor Laboratory Press, Cold Spring Harbor, N.Y., 1989; and U.S. Pat. No. 5,612,183, the entire contents of which is incorporated by reference. Exemplary hybridization conditions include, but are not limited to, incubating the fixed and permeabilized sample at 55° C. for 1.5 hours in a humidified chamber with 100-500 nM of the nucleic acid probes, followed by washing for 0.5 hours in a suitable wash buffer.

If the probe used in the drug resistance screen is specific for a certain type of microorganism, the step of identifying the microorganisms present in a clinical sample can be performed simultaneously with the drug susceptibility screen. This is an advantage of the method of the invention because it eliminates the need for two separate cultures, one for identifying the microorganisms and one for determining their drug resistance, in the methods of the prior art. For example, if the probes used in the drug resistance screen are from the S. aureus PNA FISH™ kit (AdvanDx, Woburn, Mass.), a positive signal from the drug resistance screen will indicate (1) that the microorganism present in the clinical sample is resistant to a particular drug and/or drug concentration and (2) that the clinical sample contains S. aureus.

Alternatively, if the probes used in the method of the invention are specific for a certain type of microorganism, they can be used in the optional step described above of classifying or identifying the microorganism in the clinical specimen. If the step of classifying or identifying the microorganism is performed before inoculating the subsamples into growth mediums having different antibiotic compounds and/or different antibiotic concentrations, one can select a panel of antibiotic compounds and/or concentrations suitable for testing the antibiotic resistance. For example, if a subsample of the clinical sample is treated with the probes from the S. aureus PNA FISH™ kit (AdvanDx, Woburn, Mass.), a positive signal would indicate the presence of S. aureus in the clinical sample. In that case, a panel of antibiotic compounds and/or concentrations suitable for testing the antibiotic resistance of S. aureus should be selected for the antibiotic resistance screen. Suitable panels for various types of microorganisms are known to those skilled in the art.

In some embodiments, the antibiotics and/or probes may be dried onto filters and placed at the bottom of the wells in a multiwell device. Suitable multiwell devices are known in the art and include, for example, a 96 well filter plate (Pall Corporation, New York), or the Phoenix™ NID panel (Becton Dickinson, New Jersey). The wells in the devices may be of any shape, such as square or round, and the walls of the wells may be perpendicular to the base or sloped to facilitate liquid flow. The wells may have a common filling port (e.g., Phoenix™ NID panel) or may be filled independently (e.g., Pal 96 well filter plate). The wells may have any volume capacity, for example, about 1 to 100 μl, about 5 to 50 or about 10 to 20 μl. Any suitable filters may be used, including any commercially available materials known to those skilled in the art, including, for example, track-etched polycarbonate filters (Whatman, GE) and anodisc pore filters (Whatman, GE).

In some embodiments, the filters used in the multiwell devices may comprise any number of different FISH probes and any number of antibiotics. In some embodiments, the filters may comprise about 1 to 10 different FISH probes and about 1 to 2 antibiotics. In other embodiments, the filters may comprise about 1 to 6 different FISH probes and about 1 to 2 antibiotics. In other embodiments, the filters may comprise about 1 to 3 different FISH probes and about 1 to 2 antibiotics. The filters may also comprise one or more DNA staining dyes such as PI or DAPI for distinguishing dead cells from live cells.

In some embodiments, the method of the invention is used to establish algorithms of the test system. In such embodiments, various susceptible, intermediate, or resistant strains may be tested to generate a database of information to establish various growth responses within the test system. The raw signals collected may be used to correlate with expected outcomes to establish the algorithms of the system for the interpretation of results.

In some embodiments, the test samples may include a “growth control” in which no antibiotic is added in order to collect data from a positive bacterial growth response. In some embodiments, the test samples may include a “negative control” in which no bacteria is added in order to establish a reagent “blank” baseline. In other embodiments, the test samples may include a “kill control” in which very high concentrations of the antimicrobial agent are added in order to establish a true killing response.

### EXAMPLES

### Example 1

Introduction

This experiment discloses a PNA FISH test using peptide nucleic acid probes, filter membranes, and fluorescence microscopy for the rapid and accurate detection and identification of S. aureus infections. The test results showed that the filter membrane PNA-FISH system can detect as low as 10² CFU/ml S. aureus with pure culture. The results also indicate that the method can distinguish methicillin-resistant S. aureus (MRSA) from wild type S. aureus following the incubation with Oxacillin at concentration of greater than 2.0 ug/mL.

Equipment
Water bath set to 55° C.±1° C. (Advandx Catalog No. AC006)
Staining dish with cover and slide holder (Advandx Catalog No. AC004)
Fluorescence microscope (Nikon TI)
Flow-through device

Materials
S. aureus ATCC 29213 (wild type)
S. aureus POS 3663 (MRSA)
1.5 ml microcentrifuge tubes
Phoenix ID® broth and AST® broth
S. aureus PNA FISH Kit (Advandx Catalog No. KT001)
PNA FISH microscope slides (100 pcs.) (Advandx Catalog No. AC001)
Coverslips (100 pcs.) (Advandx Catalog No. AC002)
Immersion oil
Measuring cylinders (10 mL and 500 mL)
100% Ethanol
Deionized or distilled water
Oxacillin (OX) antibiotic
0.2 μm polycarbonate membrane filters (STERLITECH lot # 177990)

Procedure for Determining the Limit of Detection

Four subsamples of the S. aureus ATCC 29213 culture were created by titrating down to 10⁵, 10⁴, 10³, and 10² CFU/ml in Phoenix ID® broth (Becton Dickinson, New Jersey). 400 μl. of each subsample was placed into separate 1.5 mL microcentrifuge tubes. The tubes were centrifuged at 5000 rpm for 3 minutes and the supernatants were discarded. The bacteria were resuspended in 200 μL of 80% ethanol and fixed at room temperature for 5 minutes. The tubes were then centrifuged at 5000 rpm for 3 minutes, and the supernatants were discarded. The bacteria were resuspended in one drop (approximately 25 μL) of the S. aureus-specific PNA-probe solution from the PNA FISH Kit and incubated for 1.5 hours at 55° C. After hybridization, 500 μL of the washing solution from the PNA FISH Kit was added and the tubes were incubated at 55° C. for 30 minutes. The tubes were centrifuged 10,000 rpm for 3 minutes and the supernatants were discarded. The bacterial pellets were resuspended in 150 μL PBS, and 40 μL of each subsample was filtered onto a 0.2 μm pore-size polycarbonate membrane. The membrane was then transferred to a glass slide and covered with a cover slip. The membrane was then imaged using a fluorescence microscope.

Procedure for the Antibiotic Susceptibility Test

S. aureus ATCC 29213 and S. aureus POS 3663 cultures with a concentration of 10⁵CFU/ml were divided into six 400 μl subsamples and incubated with or without OX antibiotic for 0 or 6 hours in Phoenix AST broth as indicated in the following table.

| | | Control | Control | OX 0 μg | OX 2 μg | OX 4 μg | OX 8 μg |
|---|---|---|---|---|---|---|---|
| | | 0 hr | 6 hr | 6 hr | 6 hr | 6 hr | 6 hr |
| Organism | Access # | incubation | incubation | incubation | incubation | incubation | incubation |
| S. aureus | ATCC 29213 | x | x | x | x | x | x |
| S. aureus | POS 3663 | x | x | x | x | x | x |

Each subsample was placed into separate 1.5 mL microcentrifuge tubes following incubation. The tubes were centrifuged at 5000 rpm for 3 minutes and the supernatants were discarded. The bacteria were resuspended in 4004 of 80% ethanol and incubated for 5 minutes at room temperature and then centrifuged at 5000 rpm for 3 minutes. The supernatants were discarded and the bacteria were resuspended in one drop (approximately 25 μL) of the S. aureus-specific PNA-probe solution from the PNA FISH Kit and incubated for 1.5 hours at 55° C. After hybridization, 500 μL of the washing solution from the PNA FISH Kit was added and the tubes were incubated at 55° C. for 30 minutes. The tubes were centrifuged 10,000 rpm for 3 minutes and the supernatants were discarded. The bacterial pellets were resuspended in 150 μL PBS, and 40 μL of each subsample was transferred onto a 0.2 μm pore-size polycarbonate membrane using a flow-through device. The membrane was then transferred to a glass slide and covered with a cover slip. The membrane was then imaged using a fluorescence microscope.

Results

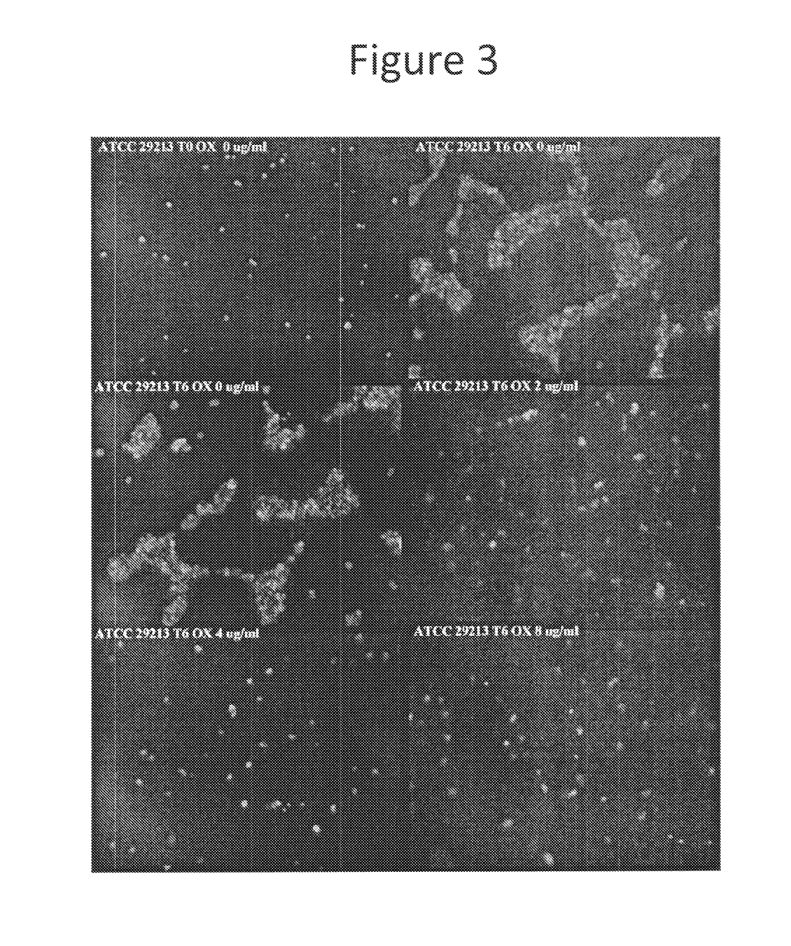

The results of the low limit of detection/identification procedure are shown in FIG. 3. These results demonstrate that a dozen stained S. aureus cells can be observed under the fluorescent microscope at concentrations as low as 10² CFU/mL.

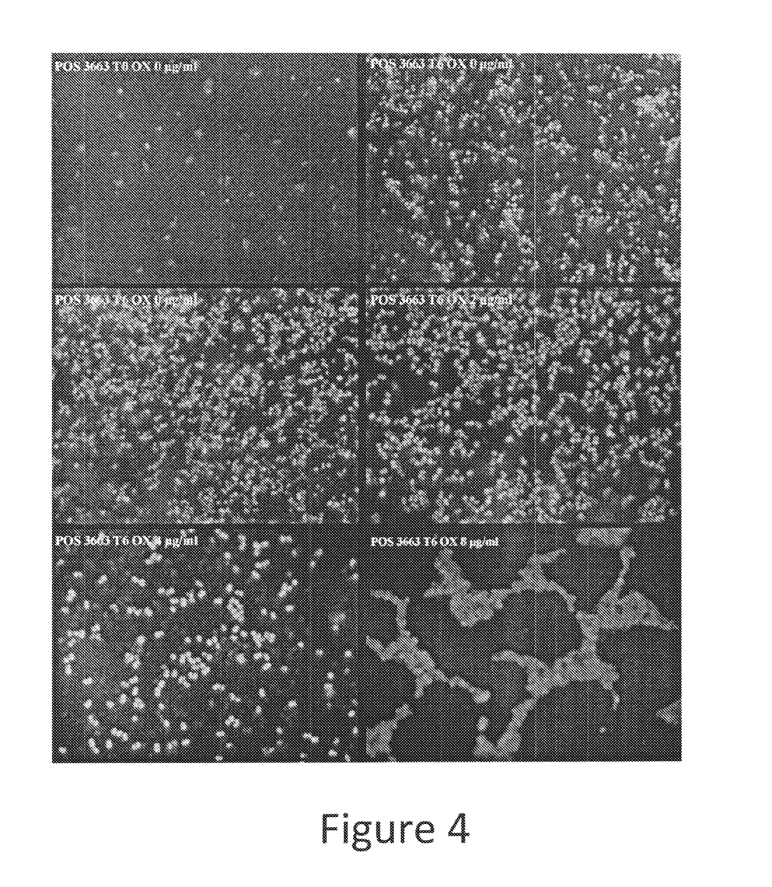

The results of the antibiotic susceptibility test are shown in FIGS. 4 and 5. FIG. 4 shows that in the absence of OX antibiotic, wild type S. aureus ATCC 29213 was identified as multiple green fluorescence clusters of cocci. In the presence of 2 μg/mL and higher concentrations of OX, most of the wild type S. aureus were killed. Dead organisms showed enlarged cells compared to live organisms. In contrast, FIG. 5 shows that S. aureus MRSA POS 3663 continues to grow in the presence of OX at concentrations as high as 8 μg/mL. In both the wild type S. aureus ATCC 29213 and MRSA POS 3663 strains, cell clumping was observed, which affected cell count and quantitation.

Summary

These experiments show that the combination of PNA-FISH, membrane filtration, and fluorescent imaging can detect at least 10² CFU/mL of microorganisms in culture samples. These experiments also show that PNA-FISH can be used to distinguish between MSRA and wild type S. aureus in a sample following incubation with oxacillin at concentrations greater than 2 μg/mL. The fact that low levels of microorganisms could be identified following only 1.5 hours of incubation eliminates the need for traditional overnight incubation followed by subsequent colony identification.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of detecting an antibiotic susceptibility of a microorganism in a sample comprising:
dividing the sample into a plurality of subsamples;
contacting each subsample with a growth medium having a different antibiotic compound and/or a different antibiotic concentration;
growing the antibiotic resistant microorganism in each antibiotic-containing subsample; and
detecting the presence of a grown antibiotic resistant microorganism in each antibiotic-containing subsample by contacting the subsample with at least one fluorescent in situ hybridization (FISH) probe that hybridizes to an antibiotic resistant microorganism,
wherein the presence of a grown antibiotic resistant microorganism in the subsample indicates that the microorganism is not susceptible to the antibiotic compound or antibiotic concentration present in the subsample, and the absence of a grown antibiotic resistant microorganism in the subsample indicates that the microorganism is susceptible to the antibiotic compound or antibiotic concentration present in the subsample.

2. The method of claim 1, wherein the sample is bronchioalveolar lavage, bronchial wash, pharyngeal exudate, tracheal aspiration, blood, serum, plasma, lymph, cerebrospinal fluid, pleural fluid, deep needle aspiration, sputum, urine, nasal secretions, tears, bile, ascites fluid, pus, synovial fluid, vitreous fluid, vaginal secretions, or urethral secretions.

3. The method of claim 1, wherein the sample is a culture fluid or specimen in which a body fluid or tissue extract from the subject has been incubated with a growth medium.

4. The method of claim 1, wherein the microorganism is a bacterium

5. The method of claim 4, wherein the microorganism is Staphylococcus, Enterococcus, Pseudomonas aeruginosa, Escherichia coli, Klebsiella pneumoniae, Acinetobacter baumannii, Streptococcus pneumoniae, Stenotrophomonas maltophilia, Burkholderia cepacia, or Ralstonia pickettii.

6. The method of claim 1, wherein the microorganism is a Methicillin Resistant Staphylococcus aureus (MRSA).

7. The method of claim 1, wherein the microorganism is a yeast.

8. The method of claim 7, wherein the yeast is Candida albicans, C. glabrata, C. tropicalis, C. krusei, C. parapsilosis, C. bracarensis, C. guilliermondii, C. lusitaniae, or C. dubliniensis.

9. The method of claim 1, wherein the antibiotic compound is amikacin, amoxicillin, amoxicillin/clavulanate, ampicillin, ampicillin/sulbactam, arbekacin, azithromycin, aztreonam, cefaclor, cefazolin, cefdinir, cefditoren, cefetamet-pivoxil, cefixime, cefmetazole, cefoperazone, cefoperazone/sulbactam, cefotaxime, cefotetan, cefotiam, cefoxitin, cefpirome, cefpodoxime-proxetil, cefsulodin, ceftazidime, ceftibuten, ceftizoxime, ceftriaxone, cefuroxime sodium, cephalexin, cephalothin, cerepime, chloramphenicol, ciprofloxacin, clarithromycin, clindamycin, colistin, daptomycin, ertapenem, erythromycin, fosfomycin, fusidic acid, garenoxacin, gatifloxacin, gemifloxacin, gentamicin, mupirocin, isepamycin, kanamycin, levofloxacin, lincomycin, linezolid, imipenem, lomefloxacin, meropenem, minocycline, moxalactam, moxifloxacin, mupirocin, nalidixic acid, netilmicin, nitrofurantoin, norfloxacin, ofloxacin, oxacillin, pefloxacin, penicillin G, piperacillin, pristinamycin, quinupristin, dalfopristin, rifampin, streptomycin, teicoplanin, telithromycin, temocillin, tetracycline, ticarcillin, ticarcillin/clavulanate, tobromycin, trimethoprim, trimethoprim/sulfamethoxazole, trimethoprim/sulfamethoxazole, or vancomycin.

10. The method of claim 1, wherein the antibiotic compound is fluconazole, itraconazole, or flucytosine.

11. The method of claim 1, wherein the FISH probe comprises a peptide nucleic acid (PNA), a locked nucleic acid (LNA), a deoxyribonucleic acid, or a ribonucleic acid.

12. The method of claim 1, wherein the FISH probe comprises a fluorophore.

13. The method of claim 1, wherein the FISH probe hybridizes to a ribosomal RNA.

14. The method of claim 1, wherein the FISH probe hybridizes to a genus or species specific nucleic acid sequence of the microorganism and thereby identifies the genus or species of the antibiotic resistant microorganism.

15. The method of claim 1, wherein a plurality of genus or species specific fluorescent in situ hybridization (FISH) probes having distinguishable labels are contacted with the subsample and the fluorescence of the FISH probe that hybridizes to the antibiotic resistant microorganism identifies the genus or species of the antibiotic resistant microorganism present in the subsample.

16. The method of claim 1, further comprising
determining the type of microorganism present in the sample, and
selecting a panel of suitable antibiotic compounds and/or different antibiotic concentrations for contacting with each subsample based upon the type of microorganism determined to be present in the sample.

17. The method of claim 16, wherein the step of determining the type of microorganism present in the sample involves Gram staining and/or hybridization to a family, genus, or species of specific fluorescent in situ hybridization (FISH) probe.

18. The method of claim 16, wherein the step of determining the type of microorganism present in the sample involves polymerase chain reaction and/or mass spectrometry.

19. The method of claim 1, wherein a series of different concentrations of an antibiotic is contacted with the microorganism in the subsamples and the minimum inhibitory concentration (MIC) of the antibiotic is determined to be the lowest antibiotic concentration that inhibits the growth of the microorganism in the subsample.

20. A method of identifying and detecting antibiotic susceptibility of a microorganism in a sample comprising:
dividing the sample into a plurality of subsamples;
identifying the type of microorganism present in one or more of the subsamples;
contacting one or more of the subsamples with a growth medium having a different antibiotic compound and/or concentration;
growing the antibiotic resistant microorganisms present in each antibiotic-containing subsample; and
detecting the presence of the grown antibiotic resistant microorganisms in each antibiotic-containing subsample by contacting the subsample with at least one fluorescent in situ hybridization (FISH) probe that hybridizes to the grown antibiotic resistant microorganisms,
wherein the presence of grown antibiotic resistant microorganisms in the subsample indicates that the microorganism is resistant to the antibiotic compound or concentration present in the subsample, while the absence of grown antibiotic resistant microorganism in the subsample indicates that the microorganism is susceptible to the antibiotic compound or concentration present in the subsample.

21. The method of claim 20, wherein the step of identifying the type of microorganism present in one or more of the subsamples involves Gram staining and/or hybridization to a family, genus, or species of specific fluorescent in situ hybridization (FISH) probe.

22. The method of claim 20, wherein the step of identifying the type of microorganism present in the sample involves polymerase chain reaction and/or mass spectrometry.

23. The method of claim 20, wherein the sample is bronchioalveolar lavage, bronchial wash, pharyngeal exudate, tracheal aspiration, blood, serum, plasma, lymph, cerebrospinal fluid, pleural fluid, deep needle aspiration, sputum, urine, nasal secretions, tears, bile, ascites fluid, pus, synovial fluid, vitreous fluid, vaginal secretions, or urethral secretions.

24. The method of claim 20, wherein the sample is a culture fluid or specimen in which a body fluid or tissue extract from the subject has been incubated with a growth medium.

25. The method of claim 20, wherein the microorganism is a bacterium.

26. The method of claim 25, wherein the microorganism is Staphylococcus, Enterococcus, Pseudomonas aeruginosa, Escherichia coli, Klebsiella pneumoniae, Acinetobacter baumannii, Streptococcus pneumoniae, Stenotrophomonas maltophilia, Burkholderia cepacia, or Ralstonia pickettii.

27. The method of claim 20, wherein the microorganism is a Methicillin Resistant Staphylococcus aureus (MRSA).

28. The method of claim 20, wherein the microorganism is a yeast.

29. The method of claim 28, wherein the yeast is Candida albicans, C. glabrata, C. tropicalis, C. krusei, C. parapsilosis, C. bracarensis, C. guilliermondii, C. lusitaniae, or C. dubliniensis.

30. The method of claim 20, wherein the antibiotic compound is amikacin, amoxicillin, amoxicillin/clavulanate, ampicillin, ampicillin/sulbactam, arbekacin, azithromycin, aztreonam, cefaclor, cefazolin, cefdinir, cefditoren, cefetamet-pivoxil, cefixime, cefmetazole, cefoperazone, cefoperazone/sulbactam, cefotaxime, cefotetan, cefotiam, cefoxitin, cefpirome, cefpodoxime-proxetil, cefsulodin, ceftazidime, ceftibuten, ceftizoxime, ceftriaxone, cefuroxime sodium, cephalexin, cephalothin, cerepime, chloramphenicol, ciprofloxacin, clarithromycin, clindamycin, colistin, daptomycin, ertapenem, erythromycin, fosfomycin, fusidic acid, garenoxacin, gatifloxacin, gemifloxacin, gentamicin, mupirocin, isepamycin, kanamycin, levofloxacin, lincomycin, linezolid, imipenem, lomefloxacin, meropenem, minocycline, moxalactam, moxifloxacin, mupirocin, nalidixic acid, netilmicin, nitrofurantoin, norfloxacin, ofloxacin, oxacillin, pefloxacin, penicillin G, piperacillin, pristinamycin, quinupristin, dalfopristin, rifampin, streptomycin, teicoplanin, telithromycin, temocillin, tetracycline, ticarcillin, ticarcillin/clavulanate, tobromycin, trimethoprim, trimethoprim/sulfamethoxazole, trimethoprim/sulfamethoxazole, or vancomycin.

31. The method of claim 20, wherein the antibiotic compound is fluconazole, itraconazole, or flucytosine.

32. The method of claim 20, wherein the FISH probe comprises a peptide nucleic acid (PNA), a locked nucleic acid (LNA), a deoxyribonucleic acid, or a ribonucleic acid.

33. The method of claim 20, wherein the FISH probe comprises a fluorophore.

34. The method of claim 20, wherein the FISH probe hybridizes to a ribosomal RNA.

35. The method of claim 20, wherein the FISH probe hybridizes to a genus or species specific nucleic acid sequence of the microorganism and thereby identifies the genus or species of the antibiotic resistant microorganism.

36. The method of claim 20, wherein a plurality of genus or species specific fluorescent in situ hybridization (FISH) probes having distinguishable labels are contacted with the subsample and the fluorescence of the FISH probe that hybridizes to the antibiotic resistant microorganism identifies the genus or species of the antibiotic resistant microorganism present in the subsample.

37. The method of claim 20, wherein a series of different concentrations of an antibiotic is contacted with the microorganism in the subsamples and the minimum inhibitory concentration (MIC) of the antibiotic is determined to be the lowest antibiotic concentration that inhibits the growth of the microorganism in the subsample.
